(19) United States
(12) Patent Application Publication
(10) Pub. No.: US 2007/0238197 A9
(43) Pub. Date: Oct. 11, 2007

(54) Fluorescent ligands for GPCR arrays

(75) Inventors: Ye Fang, Painted Post, NY (US); Yulong Hong, Painted Post, NY (US); Jinlin Peng, Painted Post, NY (US)

Correspondence Address: KAPLAN GILMAN GIBSON & DERNIER L.L.P., 900 ROUTE 9 NORTH, WOODBRIDGE, NJ, 07095, US

(73) Assignee: Corning Incorporated, Patent Dept., Corning, NY (US)

(21) Appl. No.: 11/363,400

(22) Filed: Feb. 27, 2006

Related U.S. Application Data
(62) Division of application No. 10/741,213, filed on Dec. 19, 2003, now abandoned.
(63) Continuation-in-part of application No. 10/639,718, filed on Aug. 12, 2003.
(63) Continuation-in-part of application No. 09/854,786, filed on May 14, 2001, now Pat. No. 6,977,155.
(60) Provisional application No. 60/486,592, filed on Jul. 11, 2003.
(60) Provisional application No. 60/486,592, filed on Jul. 11, 2003.

(51) Int. Cl. G01N 33/543

(52) U.S. Cl. 436/518

(57) ABSTRACT

Microarrays employing a fluorescent ligand including a material having a binding affinity in the range of about 0.01 to about 25 nM, or about 0.1 to about 10 nM; a specificity to its cognate receptor in the range of about 50 to about 99%, or about 65 to about 99%; a cross-activity to other receptors of 0 to about 20%, or 0 to about 10%; a net charge per ligand of about −3 to about +5, or more preferably, about −2 to about +2 or most preferably for small compound ligands about −1 to about +2. The ligand may also have a hydrophobicity in the range of about 3 to about 55 minutes eluting time (as measured under specified eluting conditions). In some embodiments, the ligand includes fluorescently labeled motilin 1-16 labeled with Bodipy-TMR, rhodamine or Cy5-. Other embodiments include fluorescently labeled Cy5-naltrexone, Cy5-neurotensin 2-13, N-terminal labeled neurotensin 2-13 or lys-labeled labeled neurotensin 2-13.

### CROSS REFERENCE TO RELATED APPLICATIONS

This application is a divisional application of and claims the benefit to U.S. patent application Ser. No. 10/741,213, filed Dec. 19, 2003, entitled “Fluorescent Ligands For GPCR Arrays,” the entire disclosure of which is hereby incorporated by reference, which claims benefit of priority from U.S. Provisional Application No. 60/486,592, filed on Jul. 11, 2003, the entire content of which is incorporated by reference herein, and from U.S. patent application Ser. No. 10/639,718 filed on Aug. 12, 2003, the entire content of which is incorporated by reference herein.

### BACKGROUND OF THE INVENTION

The present invention relates generally to ligand materials, and particularly to ligands suitable for use as fluorescently labeled ligands for GPCR arrays.

G-protein coupled receptors (GPCRs) represent an important class of drug targets. Approximately 50% of current drugs target GPCRs; more than $23.5 billion in pharmaceutical sales annually are ascribed to medications that address this target class. The physiological roles of GPCRs as cell-surface receptors responsible for transducing exogenous signals into intracellular responses, and the fact that the binding of natural ligands to their paired GPCR(s) can be moderated using appropriate small molecule drugs, are factors giving significant importance to drugs targeting GPCRs.

There are about 400 to 700 GPCRs in the human genome. Ligands for about 200 GPCRs have been discovered. Although there is very little conservation at the amino acid level among GPCR sequences, all the GPCRs share a characteristic motif consisting of seven distinct hydrophobic transmembrane regions, each about 20 to 30 amino acids in length, an extracellular N-terminus, and an intracellular C-terminus.

A wide range of technologies are available to screen compounds against GPCRs. An increasing pace of target identification and the increasing size of compound libraries continues to drive the development of GPCR screening technologies. These assays can be classified into cell based and GPCR-membrane based assays. The cell based assays use intact cells expressing or over-expressing a GPCR of interest. Cell based assays offer the advantage that the functional activation of GPCRs by candidate compounds can be monitored. Readout is mainly based on the generation of secondary messengers (e.g. Ca2+, cAMP, IP3, etc.). Cell based assays including reporter gene assays, β-arrestin and GPCR-GFP translocation assays (i.e., receptor internalization and endosome formation) have also been described in the literature. GPCR-membrane-based assays use membrane preparations obtained from a cell line over-expressing the receptor. Compound binding is monitored through competition assays using a fluorescent or radioactive ligand as a probe. Methods to monitor the activation of GPCRs by non-cell based assays are mostly limited to monitoring GTP-GDP exchange at the GPCR associated Ga protein using GTP analogues (35S-GTPγS or Eu-GTP). Among these technologies, fluorescence techniques have gained critical positions in the core detection technology underlying high-throughput screening systems, because of the high sensitivity of fluorescence measurements, which now extend routinely to the single molecule level. An equally important facet of the use of these techniques is the ability to use different aspects of fluorescence output (e.g., lifetime, brightness, polarization, anisotropy and energy transfer) to construct assays that do not require separation steps and that have an intrinsically higher information content. Moreover, given some simplifying assumptions, relatively straightforward formalisms can be used to describe each of these processes and allow prediction of experimental results and definition of the desired direction for future developments.

Among these fluorescence technologies, fluorescent ligand-based detection methods have gained popularity in the past several years. For example, fluorescently labeled ligands have been used to directly visualize receptor-ligand interactions with spatial and temporal resolution for cell-based assays, and to measure the binding affinity and potency of drug candidates to a GPCR using fluorescence polarization or total fluorescence intensity analysis or other methods.

GPCR microarrays can be fabricated using conventional robotic pin printing and cell membrane preparations containing GPCRs from a cell line over-expressing the receptor. We have also demonstrated assays for screening compounds using these arrays (see, for example, Fang, Y. et al. (2002) Membrane protein microarrays. J. Am. Chem. Soc. 124, 2394-2395; Fang, Y. et al. (2002) G protein-coupled receptor microarrays. Chem BioChem 3, 987-991; Fang, Y. et al. (2002) Membrane biochips. Biotechniques. 33, s62-s65; and Fang, Y. et al. (2003) G protein-coupled receptor microarrays for drug discovery. Drug Discovery Today, 8, 755-761) all of which are hereby incorporated by reference herein.

GPCR microarrays are naturally suited to analyzing multiple GPCRs simultaneously. Nevertheless, the industry has not fully realized the potentials of GPCR microarrays for drug discovery, due in part to the limited commercial availability of fluorescent ligands that are suitable for GPCR microarray applications. Although there are increasing numbers of fluorescently labeled ligands that are commercially available, these labeled ligands are not well suited for GPCR microarray applications.

What is needed, then, are fluorescently labeled ligands which have characteristics which makes them suitable for use in GPCR microarray applications.

### SUMMARY OF THE INVENTION

One aspect of the invention is a fluorescent ligand which includes a material having the following properties: a binding affinity to its cognate receptor(s) in the range of about 0.01 to about 25 nM, or more preferably about 0.1 to about 10 nM; a specificity to its cognate receptor in the range of about 50 to about 99%, or more preferably about 65 to about 99%; a cross-activity to other receptors of 0 to about 20%, or more preferably 0 to about 10% when the concentration of the ligand at 0.5˜10×Kd is used; a net charge per ligand of about −3 to about +5, or more preferably about −2 to about +2, or most preferably if the ligand is a small compound about −1 to about +2. In an alternative embodiment, the material would have a hydrophobicity in the range of about 3 to about 55 minutes (or, more preferably, about 3 to about 40 minutes) eluting time under Specified Eluting Conditions (defined below).

Another aspect of the invention is a ligand including fluorescently labeled motilin 1-16 labeled with Bodipy-TMR.

Another aspect of the invention is a ligand including fluorescently labeled motilin 1-16 labeled with rhodamine.

Another aspect of the invention is a ligand including fluorescently labeled motilin 1-16 labeled with Cy5-.

Another aspect of the invention is a ligand including fluorescently labeled Cy5-naltrexone.

Another aspect of the invention is a ligand including Cy5-neurotensin 2-13.

Another aspect of the invention is a ligand including N-terminal labeled neurotensin 2-13.

Another aspect of the invention is a ligand including lys-labeled labeled neurotensin 2-13.

Another aspect of the invention is a method of screening target compounds using a microarray, which includes the following steps: providing a plurality of receptor microspots on a substrate to form an array; contacting the array with a fluorescent labeled ligand including a material having a binding affinity in the range of about 0.01 to about 25 nM, a specificity to its cognate receptor in the range of about 50 to about 99%; a cross-activity to other receptors of 0 to about 20%; and a net charge per ligand of about −3 to about +5; and determining the binding profile of the ligand to its cognate receptor in the array.

Other aspects of the invention is the method described above wherein the receptor is a GPCR, or wherein the ligand has a eluting time in the range of about 3 to about 40 minutes under Specified Eluting Conditions (which are defined below), or where the ligand includes material chosen from the group consisting of: fluorescently labeled motilin 1-16; fluorescently labeled Cy5-naltrexone; and Cy5-neurotensin 2-13.

Embodiments of the invention provide materials which can be used as fluorescently labeled ligands which are particularly well-suited for use with GPCR arrays. Embodiments of the invention provide materials which enable robust GPCR microarray applications. Alternatively, embodiments of the invention provide materials which can also be used as fluorescently labeled ligands which are suitable for use with cell-based, and solution-based GPCR assays (i.e., fluorescence polarization assays).

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention and together with the description serve to explain the principles and operations of the invention.

### BRIEF DESCRIPTION OF THE DRAWINGS

FIG. 1 is an illustration of the structure of Cy3-motilin;

FIG. 2 is an image of human motilin receptor microarrays on a GAPS slide fabricated by Corning Incorporated after incubated with a solution containing four different labeled motilin in the absence and presence of unlabeled full length motilin at 1 μM. The images show comparisons of Bodipy-TMR-motilin 1-16, rhodamine-motilin 1-16, Cy5-motilin 1-16, and Cy3-motilin as probe ligands for human motilin receptor (MOTR) microarrays;

FIGS. 3A, 3B and 3C are illustrations of the structures of Bodipy-TMR-motilin 1-16, Cy5-motilin 1-16, and Rhodamine-motilin 1-16, respectively;

FIG. 4 is a plot of C18 reverse phase High Performance Liquid Chromatography (HPLC) profiles for Cy3-motilin, Bodipy-TMR-motilin 1-16, Cy5-motilin 1-16, and rhodamine-motilin 1-16;

FIG. 5 is an illustration of the fragmentation pattern of fluorescently labeled motilin 1-16 using mass spectroscopy;

FIGS. 6A, 6B and 6C are plots of fluorescence intensity (RFU), inhibition percentage and S/N ratios for Cy3-motilin, Bodipy-TMR-motilin 1-16, Cy5-motilin 1-16, and rhodamine-motilin 1-16;

FIGS. 7A, 7B, 7C and 7D are plots of saturation and Kd for the binding of labeled motilin 1-16 and motilin to MOTR in the microarray;

FIG. 8 is an illustration of the structures of flourescein (FL)-naltrexone and Cy5-naltrexone;

FIG. 9 illustrates two methods of two-step synthesis of Cy5-naltrexone;

FIG. 10 is a plot of HPLC profiles for FL-naltrexone and Cy5-naltrexone;

FIGS. 101A, 11B and 11C are plots of saturation curves of Cy5-naltrexone binding to opiod delta2 receptor microarrays;

FIG. 12 is an illustration of the fragmentation pattern of fluorescently labeled Cy5-neurotensin using mass spectroscopy; and

FIGS. 13A and 13B are plots of saturation and Kd for Cy5-NT2-13(lys) and Cy5-NT2-13(N-terminal), respectively.

### DETAILED DESCRIPTION OF THE PREFERRED EMBODIMENTS

Reference will now be made in detail to the present preferred embodiments of the invention, examples of which are illustrated in the accompanying drawings. Whenever possible, the same reference numerals will be used throughout the drawings to refer to the same or like parts.

An embodiment of the invention provides desired properties of a particular fluorescent ligand that is suitable for robust GPCR microarray assays. Particular embodiments of the invention include novel labeled fluorescent ligands for the detection of GPCR-ligand interactions using microarrays, particularly Bodipy-TMR- and Rhodamine-labeled motilin 1-16 for motilin receptor, Cy5-neurotensin 2-13 for neurotensin receptor subtype 1, and Cy5-naltrexone for delta2 opioid receptor.

We have carried out extensive studies to determine the desired properties of fluorescent labeled ligands which will enable robust GPCR microarray applications, and in general for surface-based assays using fluorescent ligands. Using these labeled ligands with the determined properties, a number of advantages may be achieved, including: lower non-specific binding signals to surfaces (i.e., GAPS (3-aminopropylsilane-coated) surfaces), which increases signal to noise ratios and assay sensitivity; better binding specificity and better assay robustness and reproducibility; and higher binding affinity to the receptors in the arrays.

Amine-presenting surfaces with moderate hydrophobicity (such as those known in the literature as GAPS) provide a highly desirable combination of characteristics for GPCR microarrays, including preserved lateral fluidity, high mechanical stability, and correct immobilization of receptor-membranes. Results have shown that model lipid membranes are immobilized onto GAPS with rapid kinetics, desired structures, preserved lateral fluidity, and significant mechanical stability. Ligand binding to GPCR microarrays on these surfaces is specific; binding affinities are similar to those obtained using traditional methods such as those discussed in Baker, J. G., Hall, I. P. and Hill, S. J. (2003) “Pharmacology and direct visualisation of BODIPY-TMR-CGP: a long-acting fluorescent beta(2)-adrenoceptor agonist” Brit. J. Pharmacol. 139, 232-242.

However, for bioassays using GPCR arrays on GAPS, one must into account for the surface properties for assay quality, reproducibility, and robustness. Two notable properties of GAPS surfaces, their positive charge and moderate hydrophobicity (with a water contact angle of 25˜45°), have great effect on assay design, particularly for fluorescent ligand design and selection. To minimize the non-specific binding of these ligands to GAPS surfaces, the labeled ligands should be preferably low negatively charged, neutral or positively charged with higher hydrophilicity. The neutral or positive charge would minimize the electrostatic interaction with surfaces; the higher hydrophilicity would minimize the hydrophobic interaction with surfaces.

Fluorescent ligands for GPCR microarray applications would most preferably possess the following properties:

Preferably, the ligands would be relatively hydrophilic to minimize the hydrophobic interaction with surfaces (i.e., non-specific binding to background—increasing signal-to-noise ratios), as well as lipid membranes in the microspots (i.e., reduced non-specific binding to microspots—increasing binding specificity). A hydrophobicity in the range of about 3 to about 55 minutes eluting time in C18-reverse phase HPLC (as measured using a Waters 3.9×150 mm column, part# WAT086344 (available from Waters Instruments, Inc. of Minneapolis, Minn.) under the following eluting condition: 5%-60% acetonitrile gradient in 0.1% trifluoroacetic acid aqueous solution within 60 min with 1 ml per min at room temperature) would be preferable, with an eluting time in the range of about 5 to about 40 minutes being more preferable. For the purposes of this document, these eluting conditions are hereafter referred to as “Specified Eluting Conditions”. In general, the longer the eluting time, the more hydrophobic the labeled ligand is.

Preferably, the ligands would have low net negative charges, or preferably be positively charged or neutral, to minimize the electrostatic interaction with surface (i.e., non-specific binding to background, increasing S/N ratios). A net charge per ligand in the range of about −3 to about +5 is preferable, with a range of about −2 to about +2 being more preferable. For small compound ligands (i.e., ligands in the range of ˜100 to 1500 Dalton in molecular weight; for example, naltrexone, naloxone, or CGP 12177), the range of net charge per ligand would most preferably be in the range of about −1 to about +2.

Preferably, the ligands would have good photostability to reduce the light-induced quenching (i.e., better chance for reproducibility). For example, Bodipy-TMR- or Cy dyes are more preferable than fluorescein, since the fluorescein is more sensitive to photobleaching.

Preferably, the ligands would have relatively high binding affinity (Kd) in the range of about 0.01 to about 25 nanomolar (nM), or more preferably, in the range of about 0.1 to about 10 nM. Preferably, the ligand would have a specificity to its cognate receptor in the range of about 50% to about 99%, or more preferably, in the range of about 65% to about 99%. This will lead to better assay robustness.

Preferably, the ligands would have no or minimal cross-talk to other receptors in the same microarrays (i.e., clean pharmacological profiling for compound screening), unless the same labeled ligand is specifically used for more than one receptors in the same microarrays. For example, we have found that Bodipy-TMR-CGP 12177 can be used for compound profiling against both the β1 and β2 adrenergic receptors.

In addition, smaller size of dye moiety attached is more preferred.

### EXAMPLES

The invention will be further clarified by the following examples.

### Example 1

Fluorescently Labeled Motilin 1-16 for Motilin Receptor: Motilin is a 22-amino acid peptide hormone expressed throughout the gastrointestinal tract of human and other species. The cDNA encoding the human motilin receptor (originally isolated as orphan clone GPR38) was identified in 1999 using a deorphanized approach. The amino-terminal portion of motilin, including residues 1-9, is devoid of any activity, while extension of this domain beyond the first nine residues restores binding and biological activity. Thus, the pharmacophoric domain of this hormone represents its amino-terminal decapeptide. The carboxyl-terminal region of motilin forms an α-helix that is thought to stabilize the interaction of the critical amino-terminal residues at the active site of the receptor. However, minimal length of motilin fragments that retain the high binding affinity of native molitin is motilin 1-14.

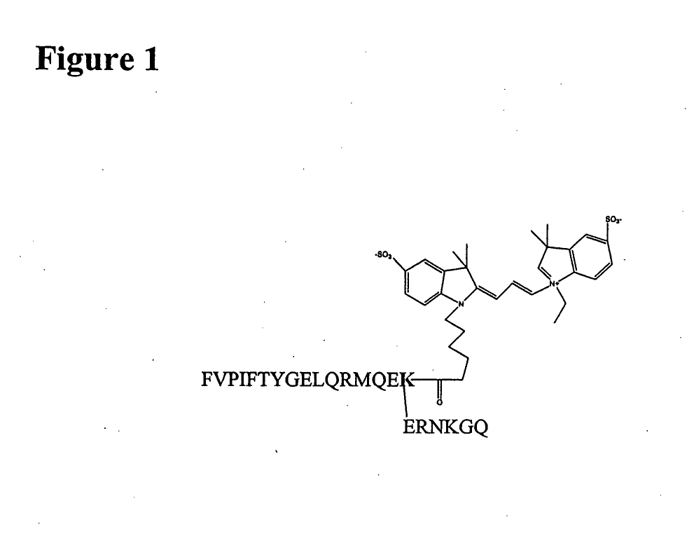

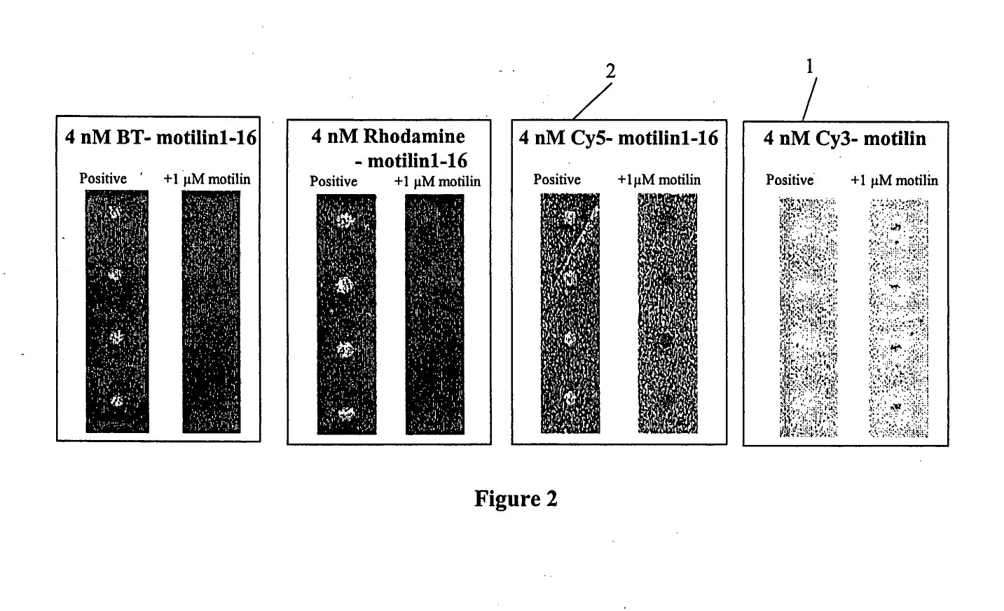

We have found out that Cy3-labeled native motilin, as illustrated in FIG. 1, gave rise to high fluorescence background with poor signal-to-noise ratio. Block 1 in FIG. 2 is an image of human motilin receptor microarrays on a GAPS slide manufactured by Corning Incorporated after interaction with Cy3-motilin at 4 nM in a binding solution in the absence (Positive) and presence (+1 μM motilin) of unlabeled motilin. Here, the Cy3-labeled motilin is used as the probe ligand for human motilin receptor arrays. The coexistence of unlabeled motilin with Cy3-motilin is used to measure the binding specificity of Cy3-motilin to the MOTR in the array. This image shows that the background due to the non-specific binding of the probes to the spare surface area is relatively high, resulting in extremely low S/N ratio.

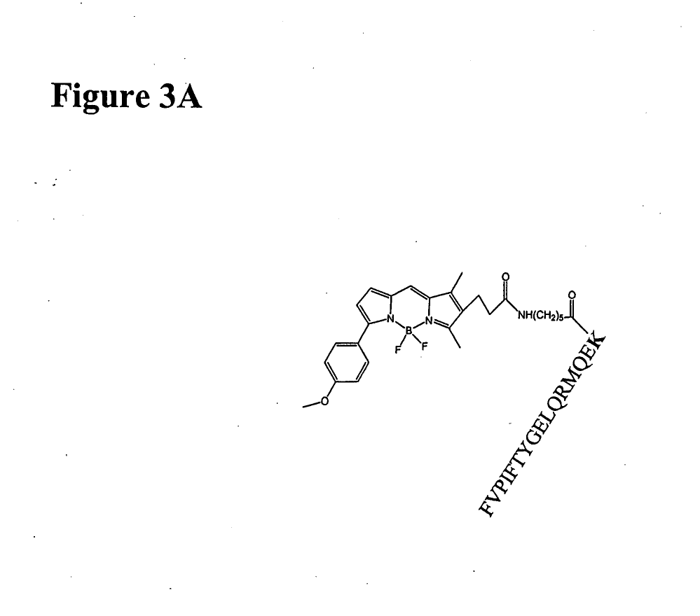

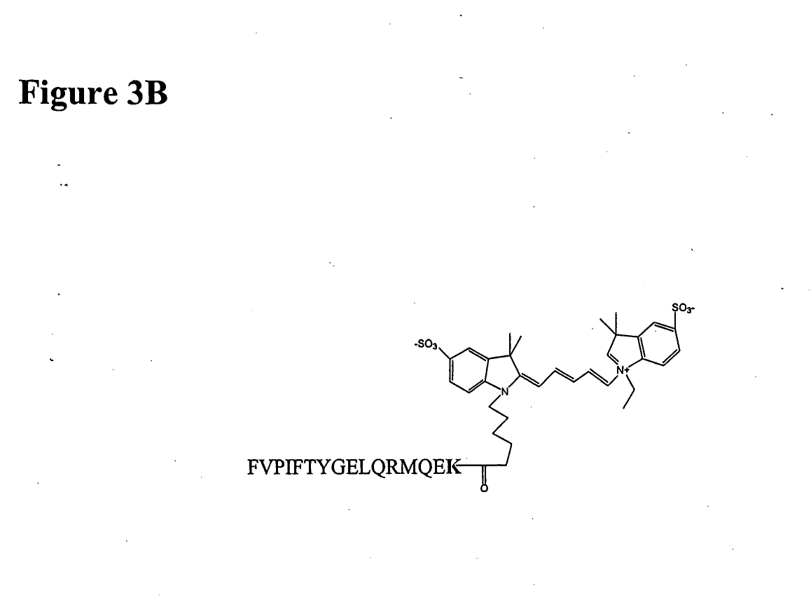

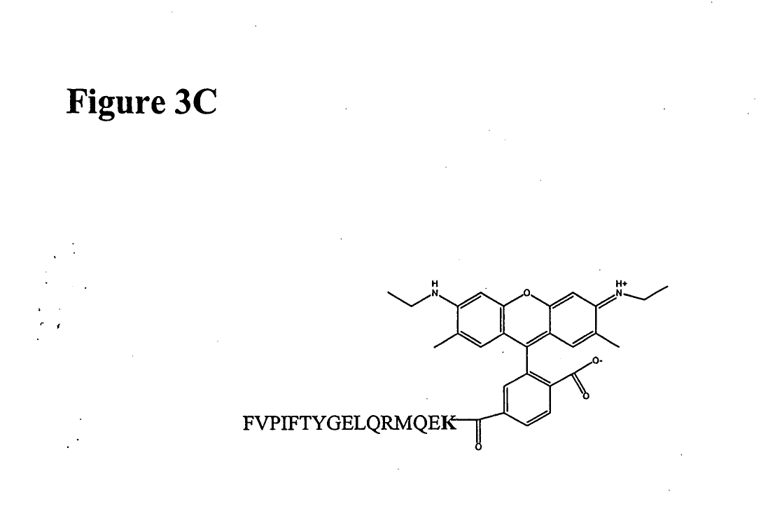

We labeled motilin 1-16 (Mot 1-16) with lysine residue at the position of 16 with three different fluorescence dyes: Bodipy-TMR-(BT), rhodamine (ROD)-, and Cy5-. The labeling was accomplished through conventional NHS ester and amine reaction. FIGS. 3A, 3B, and 3C illustrate the structures of the labeled ligands.

The fabrication of motilin receptor microarrays was carried out using a quill-pin printer (Cartesian Technologies, Model PS 5000) equipped with software for programmable aspiration and dispensing. For printing, 5-7 μL of MOTR suspension was added to different wells of a 384 well microplate. Replicate microspots were obtained using a single insertion of the pin into the solution. To prevent contamination due to carry-over between different GPCR suspensions, an automatic wash and dry cycle was incorporated. After printing, the arrays were incubated in a humid chamber at room temperature for one hour, and then used for ligand binding experiments. For the binding assays, each individual array was incubated with 10 .mu·l of a solution containing labeled ligand(s) at a particular concentration in the absence and presence of unlabeled compounds at certain concentration. The binding buffer used for all experiments was Tris-HCl (50 mM, pH 7.4) containing 10 mM MgCl2, 0.1% BSA and 1 mM EDTA.

Motilin 1-16 was obtained through custom design peptide syntheses from Sigma-Genosys. The labeled reactions were carried out through a one-step reaction by using amine-reactive fluorescent dyes from commercial vendors (i.e., Molecular Probes, Eugene, Oreg.; or Amersham Biotech, Piscataway, N.J.). The labeling reaction was done by treating solutions of the peptides in bicarbonate or phosphate buffer with solutions of N-hydroxysuccinimidyl (NHS) derivatives of the fluorescent dyes in DMSO, as recommended by these commercial vendors' protocols. The desired labeled ligands were purified using reverse phase high performance liquid chromatography (HPLC) (using an Alliance System 2690 and Nova-Pak C18 column 7.8×300 mm, Waters Inc, Milford, Mass.); the hydrophobicity of these ligands was examined using HPLC; the labeled position was examined using mass spectroscopy (using an IonSpec HiRes MALDI FT-mass spectrometer, IonSpec, Lake Forest, Calif.); the binding affinity and the cross-activity to other receptors were examined using GPCR microarrays. Results showed the following:

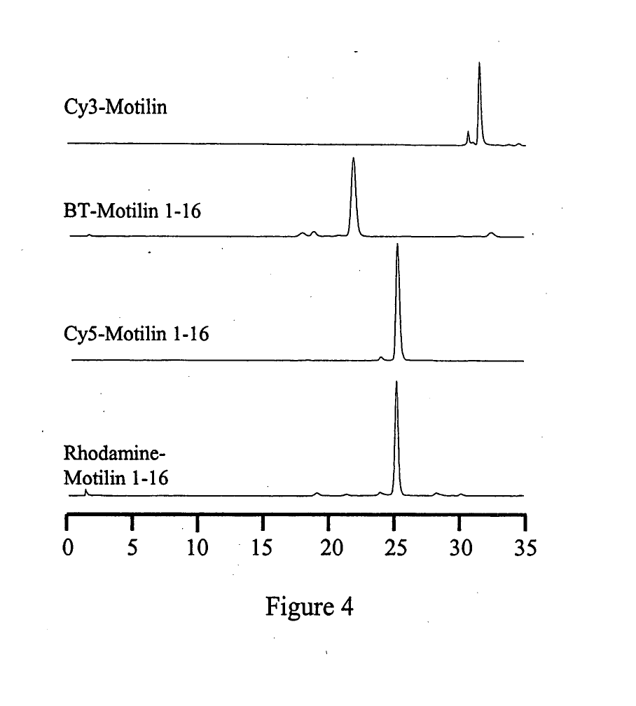

The hydrophobicity of Cy3-motilin is considerably higher than that of Cy5-motilin 1-16 and Rhodamine-motilin 1-16 (which are nearly equal), which is considerably higher than that of Bodipy-TMR (BT)-motilin 1-16. FIG. 4 shows plots of HPLC Profiles for Cy3-motilin, BT-Motilin 1-16, Cy5-motilin 1-16 and Rhodamine-motilin 1-16;

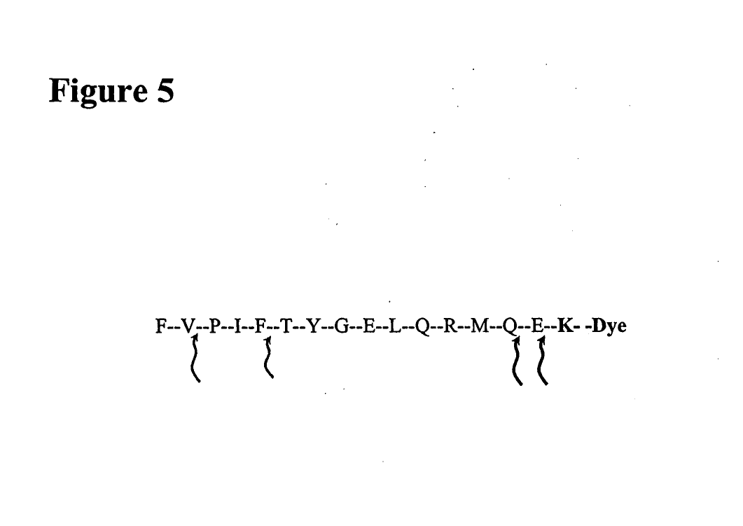

The labeled position for all four labeled ligands is located at the lysine residue at the position of 16, as confirmed by mass spectroscopy. Data from the mass spectrum analysis is given in Table 1 below, and a diagram of major fragmentation pattern is shown in FIG. 5.

TABLE 1

| | |
|---|---|
| Key m/z peaks | Assignment |

BT-motilin 1-16

| | |
|---|---|
| 2480 | BT-motilin 1-16 |
| 2233 (w) | BT-motilin 3-16 |
| 1841 | Motilin 1-15 |
| 1711 | Motilin 1-14 |
| 1406 | BT-motilin 3-16 |

Cy5-motilin 1-16

| | |
|---|---|
| 2625 | Cy5-motilin 1-16 |
| 2465 | Cy5-motilin 1-16 minus 2SO₃ |
| 2379 | Cy5-motilin 3-16 |
| 1841 | Motilin 1-15 |
| 1711 | Motilin 1-14 |

Rhodamine-motilin 1-16

| | |
|---|---|
| 2428 | ROD-motilin 1-16 |
| 2182 | ROD-motilin 3-16 |
| 1841 | Motilin 1-15 |
| 1711 | Motilin 1-14 |
| 1823 | ROD-motilin 6-16 |

The relationships between signal-to-noise ratio at 4 nM labeled ligands using MOTR
S/N(Rhodamine-motilin1-16)>S/N(BT-motilin 1-16)>>S/N(Cy5-motilin 1-16)>S/N(Cy3-motilin).

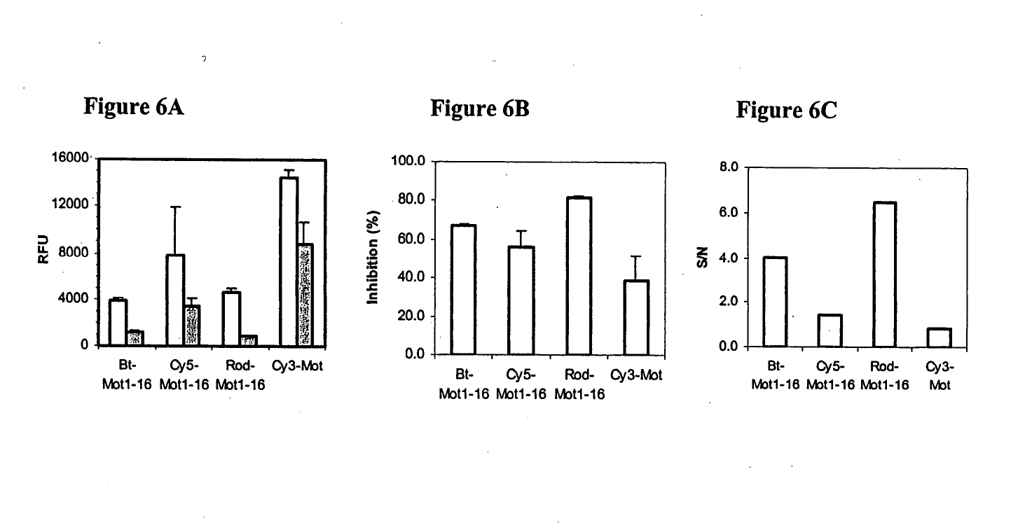

As can be seen in the images in blocks 1 and 2 in FIG. 2, the background noise due to the non-specific binding of the probe to the surface is much higher for Cy3-motilin and Cy5-motilin 1-6, therefore limiting the assay sensitivity and array performance. The non-specific binding of the probe to receptors in the microspots is higher for Cy3-motilin and Cy5-motilin 1-16, therefore limiting the assay windows and the application in high throughput screening (HTS). The difference for the performance among these probe ligands is mainly due to the size, net charge density and hydrophobicity of the labeled probes. FIGS. 6A, 6B and 6C show data plot comparison of Cy3-motilin, Rhodamine-motilin 1-16, Cy5-motilin 1-16 and BT-motilin 1-16 as the probe legands for human motilin receptor microarrays. FIG. 6A shows total signals (unshaded bar) and non-specific binding signals (shaded bar), both in terms of fluorescence intensity (RFU), after the binding of different labeled motilin to MOTR microarrays. FIG. 6B shows plots of percentage due to specific binding of the labeled ligands (i.e., inhibition percentage as a function of labeled ligands). Rhodamine- and BT-motilin 1-16 gave rise to higher binding specificity (i.e., high inhibition percentage by unlabeled motilin). FIG. 6C is a graph of signal to noise ratios, showing that signal-to-noise (S/N) ratio is much higher for rhodamine- and BT-motilin 1-16 than for Cy5- and Cy3-labeled motilin. The following formulas are used to calculate the binding specificity of labeled ligands and S/N ratio:
Binding specificity=inhibition percentage=%(100*(Iₜₒₜₐₗ−I_{non-specific})/Iₜₒₜₐₗ)
S/N ratio=Iₜₒₜₐₗ/I_{background}

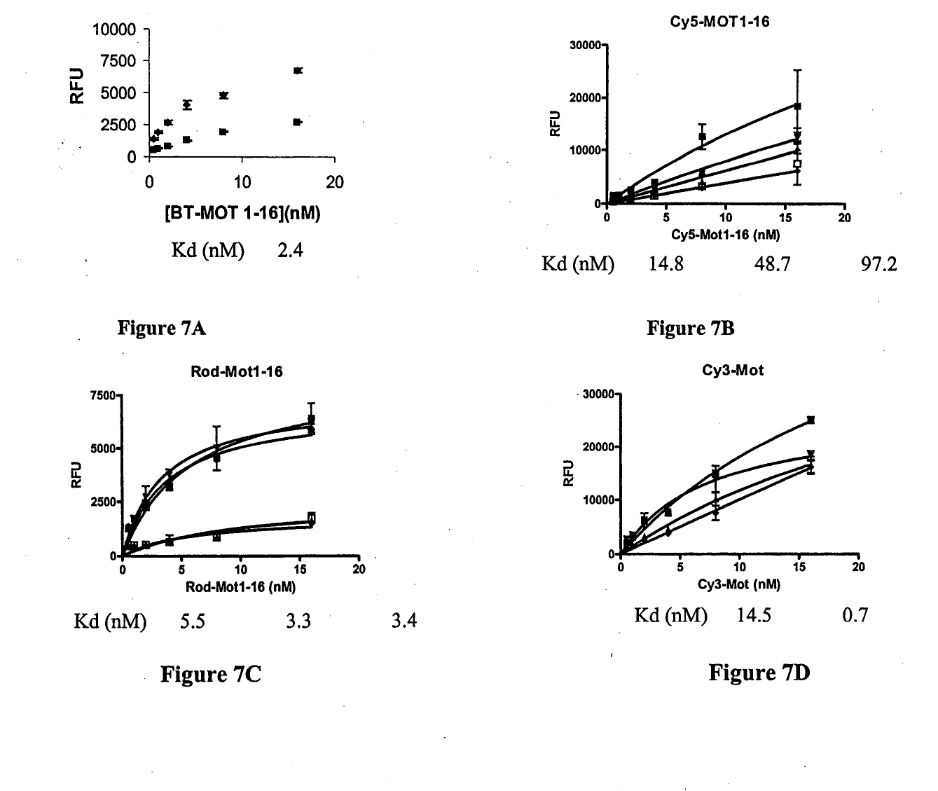

The binding affinity of BT-motilin (2.5 nM) is about the same as that of rhodamine-motilin 1-16 (3.4 nM). These are considerably greater than the binding affinity of Cy5-motilin 1-16, which is, in turn greater than the binding affinity of Cy3-motilin. FIG. 7A, 7B, 7C, and 7D are plots of saturation and Kd for the binding of labeled motilin 1-16 and motilin to MOTR microarrays. These plots show that the Kd value for BT-motilin 1-16 and rhodamine-motilin 1-16 binding to MOTR arrays is about 2.5 nm and 3.4 nM, respectively. However, due to the high non-specific binding to GAPS, the reliable binding affinities of Cy5-MOT1-16 and Cy3-MOT obtained can not be extracted from the binding data.

The binding specificity at 4 nM labeled ligands using MOTR microarrays is about the same for rhodamine-motilin1-16 and BT-motilin 1-16, and considerably less for Cy5-motilin 1-16>Cy3-motilin. This can be seen from FIGS. 6 and 7.

Both rhodamine-motilin 1-16 and BT-motilin 1-16 in the concentration range of 0.2-25 nM used shown high specificity to MOTR only, but does not bind to human neurotensin receptor subtupe 1 (NTR1), delta2 opioid receptor, beta1 adrenergic receptor, opioid-like receptor subtype 1 (ORL1), human HEK and chinese hamster ovary (CHO) control membranes (data not shown).

### Example 2

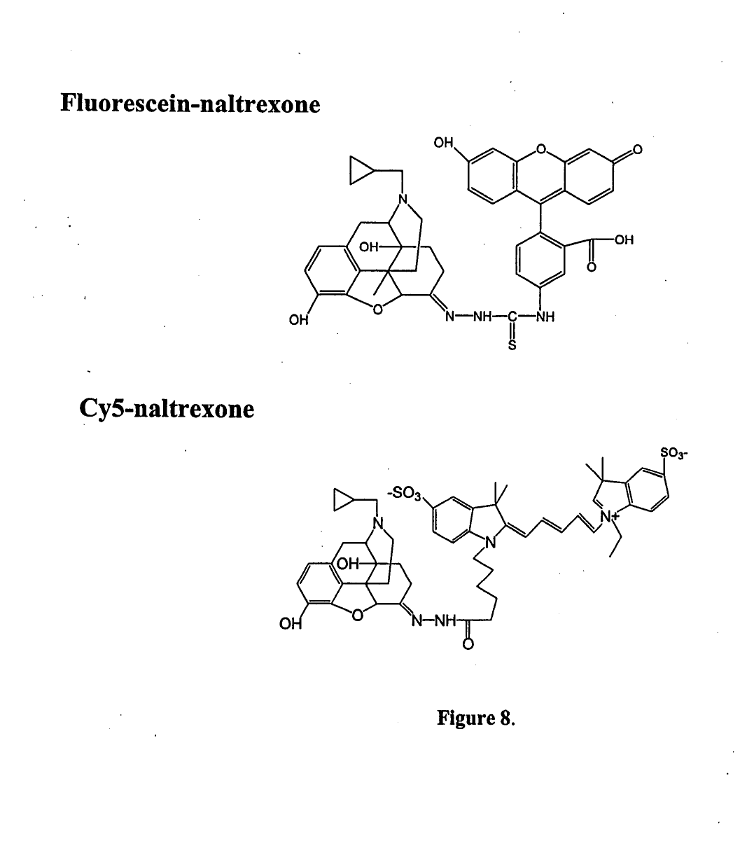

Cy5-naltrexone for Delta2 Opioid Receptor: The μ and delta2-opioid receptor plays a critical role in analgesia. One of the common antagonists that have been used to define and characterize these receptors is naltrexone, a nonaddictive drug that has been used for the treatment of opioid addiction. The fluorescent derivative, fluorescein-naltrexone, has been reported to bind to the μ-opioid binding site with high affinity, permitting their visualization in Chinese hamster ovary (CHO) cells containing transfected receptors. The fluorescein-naltrexone (FL-naltrexone), the structure of which is illustrated in FIG. 8, is commercially available from Molecular Probes, Inc. of Eugene, Oreg.

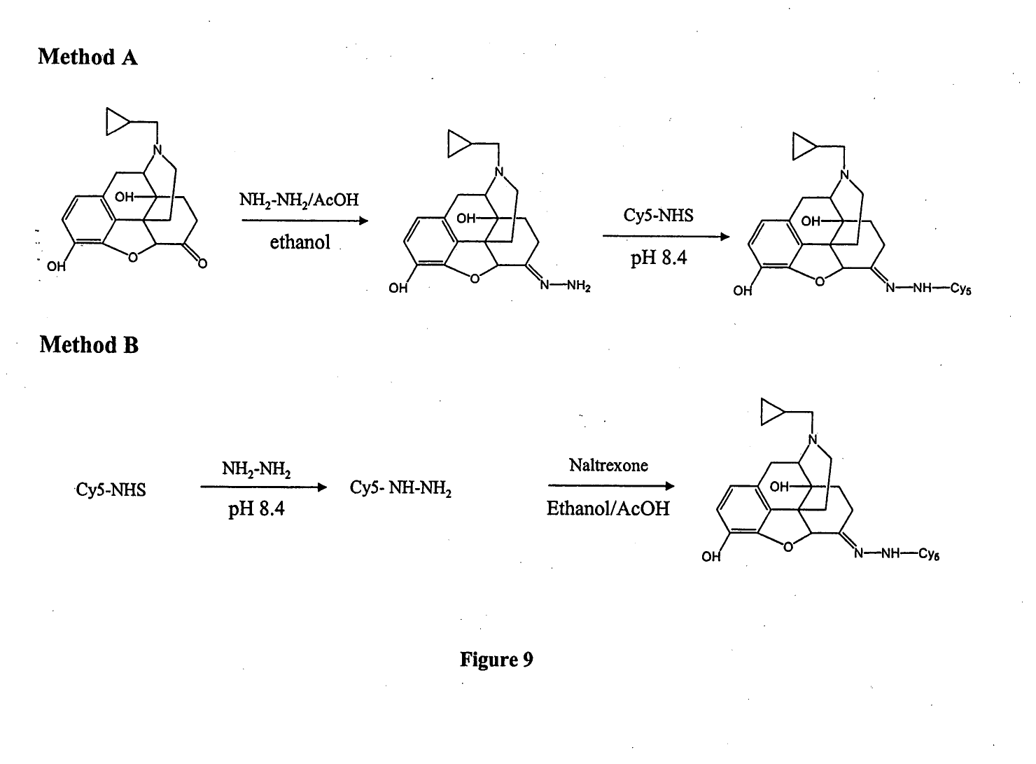

We initially used FL-naltrexone as a probe for mu and delta2 receptors in the microarrays. However, due to the poor photostability of fluorescein and the instrinitic fluorescence signals of membrane microspots in the FITC channel, we did not achieve acceptable assay performance using FL-naltrexone for opioid receptors in the microarrays and so synthesized Cy5-naltrexone using two different methods shown in FIG. 9. We found out that the method A gave rise to higher yields.

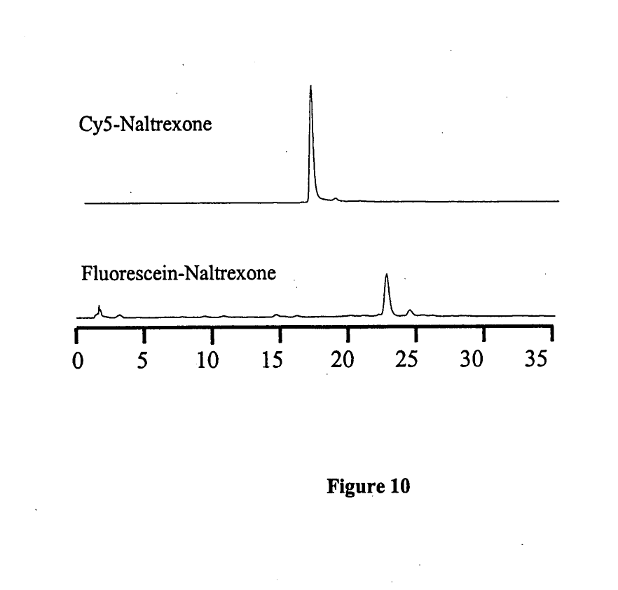

FIG. 10 is a plot of HPLC profiles showing that Cy5-naltrexone is more hydrophilic than fluorescein-naltrexone. Mass spectrum data confirmed the purity and labeled structure with a desired M/z peak at 994 (data not shown).

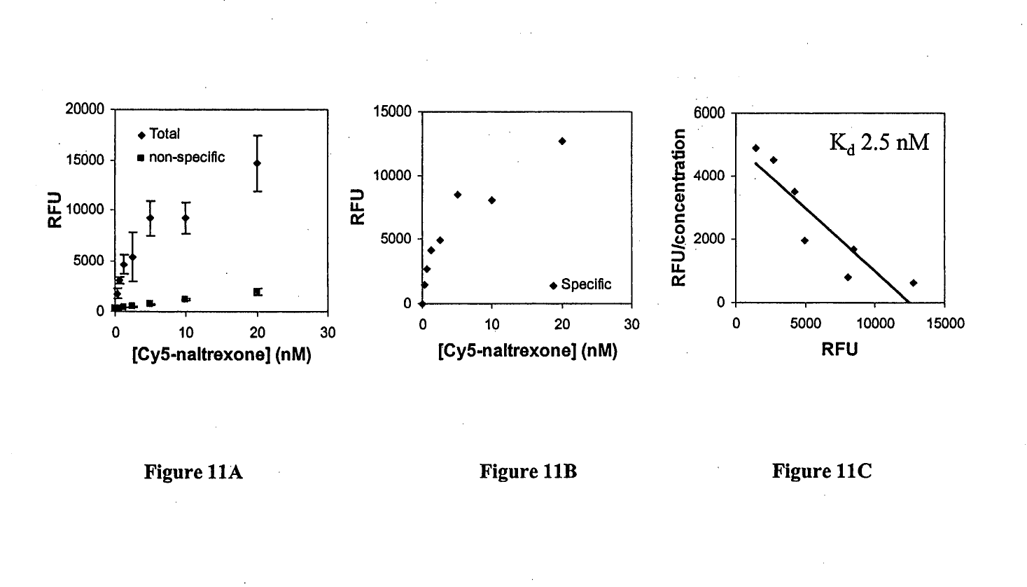

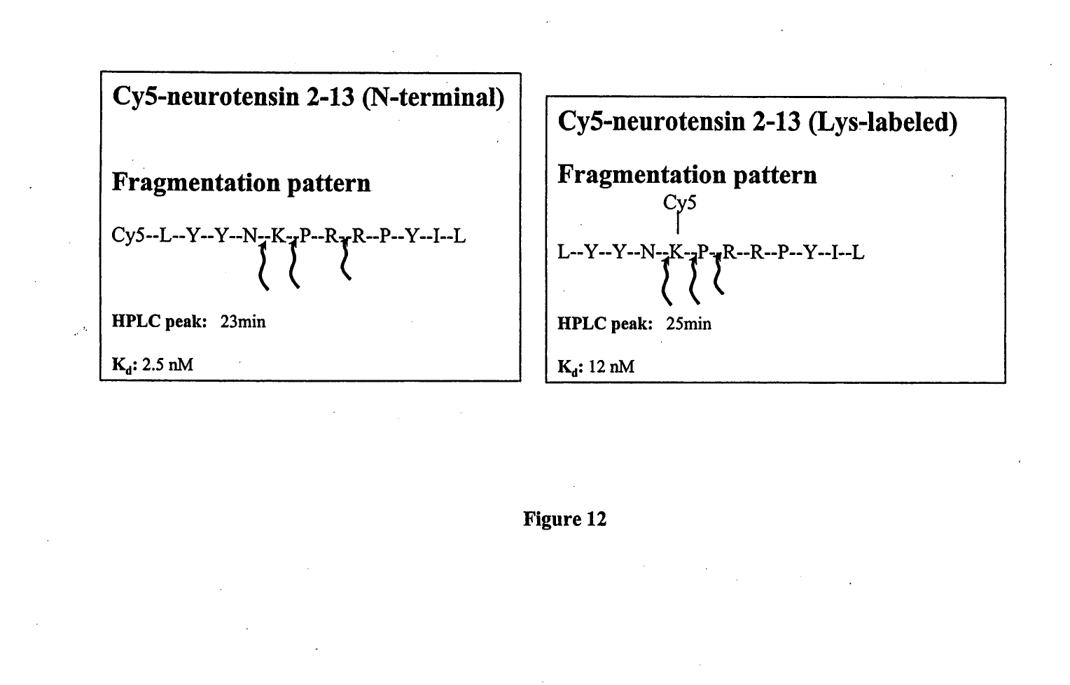

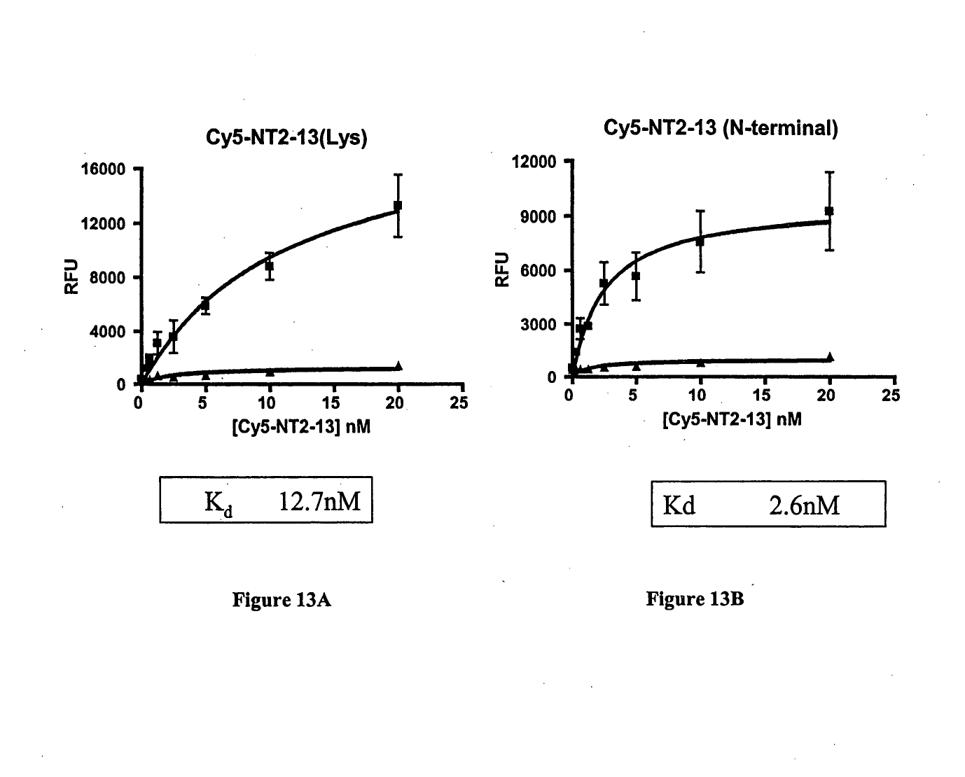

Saturation studies demonstrated that Cy5-naltrexone can bind to delta2 opioid receptor in the microarrays with a Kd of 2.5 nM (data shown in FIG. 11) and bind to mu opioid receptor in the arrays with much lower binding affinity (>15 nM) (data not shown). The Cy5-naltrexone has no across activity to NTR1, neurokinin receptor subtype II (NK2), beta1, beta2, alpha-adrenergeric receptor subtype 2A, MOTR receptors (data not shown).

### Example 3

Cy5-neurotensin 2-13 for NTR1 Receptor: Neurotensin, natural agonist of human neurotensin receptor subtype 1 (NTR1), is made up of 13 amino acids. A number of studies (see, for example, Feng H J, Zaidi J, Cusack B, et al. (2002) Synthesis and biological studies of novel neurotensin(8-13) mimetics, Bioorgan. Med. Chem. 10, 3849-3858) have shown that the last six C-terminal amino acids of this peptide are all that is needed to activate potently neurotensin receptors. Based on these studies, we have synthesized Cy5-neurotensin 2-13 by using amine-reactive fluorescent dyes from commercial vendors (i.e., Molecular Probes, Eugene, Oreg.; or Amersham Biotech, Piscataway, N.J.). The labeling reaction was done by treating solutions of the peptides in bicarbonate or phosphate buffer with solutions of N-hydroxysuccinimidyl (NHS) derivatives of the fluorescent dyes in DMSO, as recommended by these commercial vendors' protocols. The pH value of the reaction solution plays a crucial role in the labeling position and reaction yield. A pH close to 7.0 favors the covalent bond formation between the amine-reactive dye and the N-terminal NH₂ group, while a pH close to 8.5˜9.0 favors the covalent bond formation between the amine-reactive dye and the free NH₂ group of the lysine residue at the 5^{th} position of the peptide. There are two main labeled products, N-terminal labeled neurotensin 2-13 and lys-labeled neurotensin 2-13. Mass spectrum analysis data is given below in Table 2 and fragmentation patterns for these are shown in FIG. 12.

TABLE 2

| | |
|---|---|
| Key m/z peaks | Assignment |

Cy5-neurotensin 2-13 (N-terminal)

| | |
|---|---|
| 2201 | Cy5-NT2-13 |
| 2121 | Cy5-NT2-13 minus SO₃ |
| 1157 | Cy5-NT 2-5 |
| 1043 | Cy5-NT 2-4 |
| 915 | NT 7-13 |
| 643 | NT 9-13 |

Cy5-neurotensin 2-13 (Lys-labeled)

| | |
|---|---|
| 2201 (w) | Cy5-NT2-13 |
| 2121 | Cy5-NT2-13 minus SO₃ |
| 2041 | Cy5-NT2-13 minus 2SO₃ |
| 1601 | Cy5-NT6-13 minus SO₃ |
| 915 | NT 7-13 |
| 766 | Cy5-Lys |

The lysine residue is located at the position 6 of the native neurotensin, or at the position 5 of the truncated neurotensin 2-13. Results showed that the N-terminal labeled neurotensin 2-13 is slightly hydrophilic, and has higher binding affinity (i.e., lower Kd) to NTR1 in the microarrays. FIG. 14 shows a comparison of saturation and Kd for the binding of labeled neurotensin 2-13 (N-terminal labeled and Lys-labeled) to NTR1 microarrays. The Kd value of N-terminal labeled NT2-13 (2.6 nM) is lower than that of lys-labeled NT2-13 (12.7 nM). Thus, N-terminal labeled NT2-13 has a higher affinity than lys-labeled NT2-13.

The cross-activity of Cy5-neurotensin 2-13 (N-terminal) has been examined against a number of receptors; results shown that the labeled NT has no across-activity to MOTR, mu opioid receptor, delta2, beta1, beta2, a2A, ORL receptors.

It will be apparent to those skilled in the art that various modifications and variations can be made to the present invention without departing from the spirit and scope of the invention. Thus it is intended that the present invention cover the modifications and variations of this invention provided they come within the scope of the appended claims and their equivalents.

1. A system for screening target compounds comprising:
A microarray having a plurality of receptor microspots associated with a substrate;
at least one of said microspots comprising a GPCR receptor; and
a fluorescently labeled ligand for contacting said microspots comprising fluorescently labeled motilin 1-16 having a binding affinity in the range of about 0.01 to about 25 nM, a specificity to its cognate receptor in the range of about 50 to about 99%; a cross-activity to other receptors of 0 to about 20%; and a net charge per ligand of about −3 to about +5.

2. The system in accordance with claim 1, the substrate comprising glass.

3. The system in accordance with claim 1, the substrate comprising a glass surface having layer of γ-aminopropylsilane associated therewith.

4. A microarray comprising a plurality of GPCR microspots associated with a surface of a substrate coated with a γ-aminopropylsilane, the microarray adapted to receive at least one fluorescently labeled ligand for contacting said microspots comprising fluorescently labeled motilin 1-16 having a binding affinity in the range of about 0.01 to about 25 nM, a specificity to its cognate receptor in the range of about 50 to about 99%; a cross-activity to other receptors of 0 to about 20%; and a net charge per ligand of about −3 to about +5.

5. The microarray of claim 4, wherein the substrate comprises glass, metal or plastic.

6. The microarray of claim 4, wherein the substrate is configured as a chip, a slide or a microplate.

7. The ligand of claim 4 wherein said fluorescently labeled motilin 1-16 is labeled with (6-((4,4-difluoro-1,3-dimethyl-5-(4-methoxyphenyl)-4-bora-3a,4a-diaza-s-indacene-2-propionyl)amino) hexanoic acid.

8. The ligand of claim 4 wherein said fluorescently labeled motilin 1-16 is labeled with rhodamine.

9. The ligand of claim 4 wherein said fluorescently labeled motilin 1-16 is labeled with Cy5[-].
